(19) United States
(12) Patent Application Publication
(10) Pub. No.: US 2011/0106110 A1
(43) Pub. Date: May 5, 2011

(54) DEVICES AND METHODS FOR IMPLANTING A PLURALITY OF DRUG DEPOTS HAVING ONE OR MORE ANCHORING MEMBERS

(75) Inventor: William F. McKay, Memphis, TN (US)

(73) Assignee: Warsaw Orthopedic, Inc., Warsaw, IN (US)

(21) Appl. No.: 12/609,934

(22) Filed: Oct. 30, 2009

(51) Int. Cl. A61B 17/00; A61M 37/00

(52) U.S. Cl. 606/144; 604/57; 604/500

(57) ABSTRACT

The present invention is directed to a device for implanting a plurality of drug depots at or near a target tissue site beneath the skin of a patient. The device comprises at least three or more drug depots, wherein each of the at least three or more drug depots has a first surface adapted to receive one or more anchoring members so as to limit movement of the at least three or more drug depots at or near the target tissue site, and wherein at least two of the at least three or more drug depots comprise a second surface adapted to receive the anchoring member after the anchoring member contacts the target tissue site. Each drug depot is capable of releasing a therapeutically effective amount of a drug over a period of at least one day.

### BACKGROUND OF THE INVENTION

Drugs may be delivered to patients by a variety of methods including oral, intravenous, intramuscular, inhalation, topical, subcutaneous delivery or delivery directly or locally to the treatment site (e.g., intrathecally, intraspinally, intraarticularly, etc.). The method of delivery chosen depends, among other things, upon the condition being treated, the desired therapeutic concentration of the drug to be achieved in the patient and the duration of drug concentration that must be maintained.

Recently, drug depots have been developed which allow a drug to be introduced or administered to sites beneath the skin of a patient so that the drug is slowly released over a long period of time. Such drug depots allow the drug to be released from the depot in a relatively uniform dose over weeks, months or even years. This method of administering drugs is becoming especially important for contraceptives and cancer drugs that are implanted subcutaneously.

Sometimes, after a drug depot is implanted at a treatment site, the drug depot may migrate from the implant site prior to surgical closure (e.g., floats off in blood or shifts as tissues are repositioned during surgical site closure) or as physiological conditions change (e.g., repair and regeneration of cells, tissue ingrowth, movement at implant site, etc.). At times, this may reduce efficacy of the drug as the drug depot migrates away from the implant site and lodges in a distant site. If this occurs, the drug depot will have to be removed from the distant site and have to be reinserted causing additional physical and psychological trauma to a patient. Migration of the drug depot could also result in it being sucked up into a surgical wound drain or plugging it up. In some cases, if the drug depot migrates into a joint, the drug depot may inhibit movement. In more severe cases, if the drug depot migrates, it may restrict blood flow causing an ischemic event (e.g., embolism, necrosis, infarction, etc.), which could be detrimental to the patient. When multiple depots are implanted at a treatment site, the drug depots may migrate closer together (possibly on top of each other) or further apart which may reduce the clinical efficacy of the multiple depots or cause adverse events by having a high concentration of polymer degradation products. Evenly spacing the drug depots from one another allows for the body to more efficiently and safely resorb the degradable polymer.

Post-operative pain tends to be a difficult condition to treat and may be detrimental to the patient if not properly treated. Post-operative pain may be a result of the surgery, or other treatments such as, for example, management of acute pain following burns or non-surgical trauma. The goal for post-operative pain management is to reduce or eliminate pain and discomfort with medication that causes minimum or no side effects.

The site of the surgery has a profound effect upon the degree of postoperative pain a patient may suffer. In general, operations on the thorax and upper abdomen are more painful than operations on the lower abdomen, which in turn are more painful than peripheral operations on the limbs. However, any operation involving a body cavity, large joint surfaces, the spine or deep tissues should be regarded as painful. In particular, operations on the thorax or upper abdomen may produce widespread changes in pulmonary function, an increase in abdominal muscle tone and an associated decrease in diaphragmatic function. The result will be an inability to cough and clear secretions, which may lead to lung collapse and pneumonia. Prolonged pain can reduce physical activity and lead to venous stasis and an increased risk of deep vein thrombosis and consequently pulmonary embolism. In addition, there can be widespread effects on gut and urinary tract motility, which may lead in turn to postoperative ileus, nausea, vomiting and urinary retention. These problems are unpleasant for the patient, may prolong the hospital stay and are exacerbated if after implantation, a drug depot migrates away from the implant site.

New drug depot compositions and methods are needed which can easily allow accurate and precise placement of drug depots while maintaining the drug depots at implant sites and effectively treating post-operative pain.

### SUMMARY OF THE INVENTION

New implantable devices that improve drug efficacy and reduce unwanted migration of a drug are provided. In various embodiments, new implantable devices and methods are provided that effectively prevent, treat and/or reduce postoperative pain and/or inflammation by providing consistent analgesic and/or anti-inflammatory efficacy at the target tissue site of pain generation. In various embodiments, devices comprising multiple drug depots pre-attached to a suture with pre-attached needles at its ends are provided wherein the suture is also pre-knotted, which easily allows for a surgeon to suture the device to the target tissue site of pain generation.

In one exemplary embodiment, a device for implanting a plurality of drug depots at or near a target tissue site beneath the skin of a patient is provided. The device comprises at least three or more drug depots which each have a first surface adapted to receive one or more anchoring members so as to limit movement of the three or more drug depots at or near the target tissue site. At least two of the three or more drug depots comprise a second surface adapted to receive the anchoring member after the anchoring member contacts the target tissue site. The drug depots are each capable of releasing a therapeutically effective amount of the drug over a period of at least 1 day, at least 3 days, at least 7 days, 7-10 days or 3-30 days to treat post-operative pain.

In some embodiments, the first surface and the second surface of the drug depots comprise one or more channels, holes, ports, grooves, slits, loops, hooks, barbs, posts and/or clips adapted to receive the one or more anchoring members. Further, in various embodiments, the drug depots have a body and the one or more channels, holes, ports, grooves, slits, loops, hooks, barbs, posts and/or clips extend from the body of each drug depot.

In some embodiments, the one or more anchoring members may comprise one or more biodegradable sutures, yarns, threads, lines, staples and/or tacks.

The drug depots in various embodiments may be biodegradable and the first and second surface may comprise one or more channels or holes adapted to receive a biodegradable suture. The drug depots may be evenly spaced from each other on the biodegradable suture to provide uniform release of the drug from the device. The biodegradable suture may comprise a proximal end and a distal end wherein the proximal end is attached to a first needle for piercing tissue at or near the target tissue site and the distal end is attached to a second needle for piercing tissue at or near the target tissue site. The biodegradable suture may further comprise a region that surrounds the one or more channels or holes and prevents each drug depot from moving closer or farther from each other. The region of the suture may comprise a knot, rim, bead, spacer, or clip that prevents each drug depot from moving closer or farther from each other. Alternatively, the biodegradable suture may comprise one or more of a knot, rim, bead or clip disposed near the proximal end and the distal end before the first and second needles wherein the one or more of a knot, rim, bead or clip are configured to pass through tissue and the one or more channels or holes of the second surface to hold the at least two drug depots in position at the target tissue site.

In some embodiments, at least one of the drug depots has a first surface adapted to receive one or more anchoring members so as to limit movement, but not a second surface adapted to receive the anchoring member after the anchoring member contacts the target tissue site.

In various embodiments, each drug depot comprises an analgesic and/or anti-inflammatory agent or pharmaceutically acceptable salts thereof and each drug depot is biodegradable and adapted to release the drug over a period of at least 1 day, at least 3 days, at least 7 days, 7-10 days or 3-30 days to treat post-operative pain.

In another exemplary embodiment, a device for implanting a plurality of drug depots at or near a target tissue site beneath the skin of a patient is provided wherein the device comprises at least three or more drug depots, each of the at least three or more drug depots has a first channel or hole adapted to receive one or more sutures so as to limit movement of the at least three or more drug depots at or near the target tissue site, and at least two of the at least three or more drug depots comprise a second channel or hole adapted to receive a suture after the suture contacts the target tissue site. Each drug depot is capable of releasing a therapeutically effective amount of a drug over a period of at least 1 day, at least 3 days, at least 7 days, 7-10 days or 3-30 days to treat post-operative pain. Each drug depot may be biodegradable and the first channel or hole may receive a biodegradable suture and the second channel or hole may receive the same biodegradable suture after the suture contacts the target tissue site so as to anchor the device at or near a target tissue site wherein at least one drug depot does not contain a second channel or hole and each drug depot is evenly spaced from each other on the biodegradable suture to provide uniform release of the drug from the device. The suture may comprise a proximal end and a distal end such that the proximal end is attached to a first needle for piercing tissue at or near a target tissue site and the distal end is attached to a second needle for piercing tissue at or near a target tissue site. The suture may comprise a pair of second regions, each being larger than the second channels or holes of the depots. One of the second regions of the suture is adapted to pass through the second channel or hole of the depot closest to the proximal part of the suture when a sufficient pulling force is applied to pull the second region through the second hole or channel of that depot to limit movement of the device at the target tissue site. The other second region of the suture is adapted to pass through the second channel or hole of the depot closest to the distal part of the suture when a sufficient pulling force is applied to pull the second region through the second hole or channel of that depot to limit movement of the device at the target tissue site.

In some embodiments, a method of treating or preventing postoperative pain or inflammation in a patient in need of such treatment is provided wherein the method comprises suturing a device at or near the target tissue site. The device comprises at least three or more drug depots wherein each of the three or more drug depots has a first channel or hole that receives a suture, at least two of the three or more drug depots comprise a second channel or hole adapted to receive the suture after the suture contacts the target tissue and each drug depot is capable of releasing an analgesic and/or anti-inflammatory agent or pharmaceutically acceptable salt thereof over a period of at least 1 day, at least 3 days, at least 7 days, 7-10 days or 3-30 days. The suture may further comprise a pair of second regions which are larger than the second channels or holes of the depots. One of the second regions of the suture is adapted to pass through the second channel or hole of a depot closest to the proximal part of the suture when a sufficient pulling force is applied to pull the second region through the second hole or channel of that depot to limit movement of the device at the target tissue site. The other second region of the suture is adapted to pass through the second channel or hole of a depot closest to the distal part of the suture when a sufficient pulling force is applied to pull the second region through the second hole or channel of that depot to limit movement of the device at the target tissue site.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 is a front view of one embodiment of an implantable device having three drug depots connected by a suture comprising first and second needles for piercing tissue at or near a target tissue site.

FIG. 2 is a front view of another embodiment of an implantable device having three drug depots connected by a suture comprising first and second needles for piercing tissue at or near a target tissue site.

FIG. 3 illustrates a front view of a drug depot of an implantable device after a suture and needle have passed through the second channel.

FIG. 4 illustrates a front view of the drug depot shown in FIG. 3 as the suture is pulled through the second channel. The surgeon pulls the suture through the second channel to lock the depot (and ultimately the device) in place at or near a target tissue site.

FIG. 5 illustrates a back view of the drug depot shown in FIG. 4 after the suture has been pulled through the second channel.

FIG. 6 is a front view of another embodiment of an implantable device having three drug depots connected by a suture comprising first and second needles for piercing tissue at or near a target tissue site.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding the numerical ranges and parameters set forth herein and the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

The headings below are not meant to limit the disclosure in any way; embodiments under any one heading may be used in conjunction with embodiments under any other heading.

### DEFINITIONS

It is noted that, as used in this specification and the appended claims, the singular forms “a,” “an” and “the” include plural referents unless expressly and unequivocally limited to one referent. Thus, for example, reference to “a drug depot” includes one, two, three or more drug depots.

“Analgesic” refers to an agent or compound that can reduce, relieve or eliminate pain. Examples of analgesic agents include but are not limited to acetaminophen, a local anesthetic, such as for example, lidocaine, bupivicaine, ropivacaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, levorphanol, mepiridine, methadone, morphine, nalbuphine, opium, oxycodone, papaveretum, pentazocine, pethidine, phenoperidine, piritramide, dextropropoxyphene, remifentanil, sufentanil, tilidine, tramadol, codeine, dihydrocodeine, meptazinol, dezocine, eptazocine, flupirtine or a combination thereof.

The phrase “anti-inflammatory agent” refers to an agent or compound that has anti-inflammatory effects. These agents may remedy pain by reducing inflammation. Examples of anti-inflammatory agents include but are not limited to a statin, sulindac, sulfasalazine, naroxyn, diclofenac, indomethacin, ibuprofen, flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, clonidine, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate or a combination thereof. Anti-inflammatory agents also include other compounds such as steroids, for example, fluocinolone, cortisol, cortisone, hydrocortisone, fludrocortisone, prednisone, prednisolone, methylprednisolone, triamcinolone, betamethasone, dexamethasone, beclomethasone, fluticasone interleukin-1 receptor antagonists, thalidomide (a TNF-α release inhibitor), thalidomide analogues (which reduce TNF-α production by macrophages), bone morphogenetic protein (BMP) type 2 or BMP-4 (inhibitors of caspase 8, a TNF-α activator), quinapril (an inhibitor of angiotensin II, which upregulates TNF-α), interferons such as IL-11 (which modulate TNF-α receptor expression), and aurintricarboxylic acid (which inhibits TNF-α), guanidinoethyldisulfide or a combination thereof.

Exemplary anti-inflammatory agents include, for example, naproxen; diclofenac; celecoxib; sulindac; diflunisal; piroxicam; indomethacin; etodolac; meloxicam; ibuprofen; ketoprofen; r-flurbiprofen; mefenamic; nabumetone; tolmetin, and sodium salts of each of the foregoing; ketorolac bromethamine; ketorolac tromethamine; ketorolac acid; choline magnesium trisalicylate; rofecoxib; valdecoxib; lumiracoxib; etoricoxib; aspirin; salicylic acid and its sodium salt; salicylate esters of alpha, beta, gamma-tocopherols and tocotrienols (and all their d, l, and racemic isomers); methyl, ethyl, propyl, isopropyl, n-butyl, sec-butyl, t-butyl, esters of acetylsalicylic acid; tenoxicam; aceclofenac; nimesulide; nepafenac; amfenac; bromfenac; flufenamate; phenylbutazone or a combination thereof.

Exemplary steroids include, for example, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone 21-acetate, dexamethasone 21-phosphate di-Na salt, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide or a combination thereof.

Examples of a useful statin for treatment of pain and/or inflammation include but are not limited to atorvastatin, simvastatin, pravastatin, cerivastatin, mevastatin (see U.S. Pat. No. 3,883,140, the entire disclosure is herein incorporated by reference), velostatin (also called synvinolin; see U.S. Pat. Nos. 4,448,784 and 4,450,171, these entire disclosures are herein incorporated by reference), fluvastatin, lovastatin, rosuvastatin and fluindostatin (Sandoz XU-62-320), dalvastain (EP Appln. Publn. No. 738510 A2, the entire disclosure is herein incorporated by reference), eptastatin, pitavastatin, or pharmaceutically acceptable salts thereof or a combination thereof. In various embodiments, the statin may comprise mixtures of (+)R and (−)-S enantiomers of the statin. In various embodiments, the statin may comprise a 1:1 racemic mixture of the statin. Anti-inflammatory agents also include those with anti-inflammatory properties, such as, for example, amitriptyline, carbamazepine, gabapentin, pregabalin, clonidine or a combination thereof.

Unless otherwise specified or apparent from context, where this specification and the set of claims that follow refer to a drug (e.g., an anti-inflammatory agent, analgesic, and the like), the inventor is also referring to a pharmaceutically acceptable salt of the drug including stereoisomers. Pharmaceutically acceptable salts include those salt-forming acids and bases that do not substantially increase the toxicity of the compound. Some examples of potentially suitable salts include salts of alkali metals such as magnesium, calcium, sodium, potassium and ammonium, salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids or the like.

“Treating” or treatment of a disease or condition refers to executing a protocol, which may include administering one or more drugs to a patient (human, normal or otherwise, or other mammal) in an effort to alleviate signs or symptoms of the disease. Alleviation can occur prior to signs or symptoms of the disease or condition appearing as well as after their appearance. Thus, “treating” or “treatment” includes “preventing” or “prevention” of disease or undesirable condition. In addition, “treating” or “treatment” does not require complete alleviation of signs or symptoms, does not require a cure and specifically includes protocols that have only a marginal effect on the patient. “Reducing pain” includes a decrease in pain and does not require complete alleviation of pain signs or symptoms and does not require a cure. In various embodiments, reducing pain includes even a marginal decrease in pain. By way of example, the administration of the effective dosages of at least one analgesic agent and at least one anti-inflammatory agent may be used to prevent, treat or relieve the symptoms of pain and/or inflammation.

“Localized” delivery includes delivery where one or more drug depots are deposited within a tissue, for example, a nerve root of the nervous system or a region of the brain, or in close proximity (within about 10 cm, or preferably within about 5 cm, for example) thereto. A “targeted delivery system” provides delivery of one or more drug depots having a quantity of therapeutic agent that can be deposited at or near the target site as needed for treatment of pain, inflammation or other disease or condition.

The term “mammal” refers to organisms from the taxonomy class “mammalian” including but not limited to humans, other primates such as chimpanzees, apes, orangutans and monkeys, rats, mice, cats, dogs, cows, horses, etc. In various embodiments, the mammal is a human patient.

### Drug Depots

In some embodiments, a device comprising at least three or more drug depots implantable at or near a target tissue site beneath the skin of a patient is provided. The drug depots each have a first surface adapted to receive one or more anchoring members so as to limit movement of the three or more drug depots at or near the target tissue site. At least two of the three or more drug depots comprise a second surface adapted to receive the anchoring member after the anchoring member contacts the target tissue site. The drug depots are each capable of releasing a therapeutically effective amount of the drug over a period of at least 1 day, at least 3 days, at least 7 days, 7-10 days or 3-30 days.

A “drug depot” comprises a composition in which at least one active pharmaceutical ingredient or drug is administered to the body. Thus, each drug depot may comprise a physical structure to facilitate implantation and retention in a desired site (e.g., a disc space, a spinal canal, a tissue of the patient, particularly at or near a site of surgery, pain, or site of inflammation, etc.). Each drug depot also comprises the drug itself. The term “drug” as used herein is generally meant to refer to any substance that alters the physiology of a patient. The term “drug” may be used interchangeably herein with the terms “therapeutic agent,” “therapeutically effective amount,” and “active pharmaceutical ingredient” or “API.” It will be understood that unless otherwise specified, a “drug” formulation may include more than one therapeutic agent, wherein exemplary combinations of therapeutic agents include a combination of two or more drugs. The drug provides a concentration gradient of the therapeutic agent for delivery to the site. In various embodiments, each drug depot provides an optimal drug concentration gradient of the therapeutic agent at a distance of up to about 0.1 cm to about 5 cm from the implant site and comprises at least one anti-inflammatory agent or its pharmaceutically acceptable salt and/or at least one analgesic agent or its pharmaceutically acceptable salt.

A “depot” includes but is not limited to capsules, coatings, matrices, wafers, sheets, strips, ribbons, pills, pellets or other pharmaceutical delivery forms or a combination thereof. Suitable materials for the depots are ideally pharmaceutically acceptable biodegradable and/or any bioabsorbable materials that are preferably FDA approved or GRAS materials. These materials can be polymeric or non-polymeric, as well as synthetic or naturally occurring or a combination thereof. Typically, the depots will be a solid or semi-solid formulation comprised of a biocompatible material that can be biodegradable. The term “solid” is intended to mean a rigid material, while “semi-solid” is intended to mean a material that has some degree of flexibility, thereby allowing the depots to bend and conform to the surrounding tissue requirements.

A “therapeutically effective amount” or “effective amount” is such that when the device is administered, the drug results in alteration of the biological activity, such as, for example, inhibition of inflammation, reduction or alleviation of pain, improvement in the condition through muscle relaxation, etc. The dosage administered to a patient can unless otherwise specified or apparent from context be as single or multiple doses depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired. In some embodiments, the formulation is designed for immediate release. In other embodiments, the formulation is designed for sustained release. In other embodiments, the formulation comprises one or more immediate release surfaces and one or more sustain release surfaces.

The phrases “sustained release” or “sustain release” (also referred to as extended release or controlled release) are used herein to refer to one or more therapeutic agent(s) that are introduced into the body of a human or other mammal and continuously or continually releases a stream of one or more therapeutic agents over a predetermined time period and at a therapeutic level sufficient to achieve a desired therapeutic effect throughout the predetermined time period. Reference to a continuous or continual release stream is intended to encompass release that occurs as the result of biodegradation in vivo of the drug depot, or a matrix or component thereof, or as the result of metabolic transformation or dissolution of the therapeutic agent(s) or conjugates of therapeutic agent(s). As persons of ordinary skill are aware, sustained release formulations may, by way of example, be created as films, slabs, pellets, microparticles, microspheres, microcapsules, spheroids, shaped derivatives and paste. Further, the formulations may be used in conjunction with any implantable, or insertable system that a person of ordinary skill would appreciate as useful in connection with embodiments herein including but not limited to parenteral formulations, microcapsules, pastes, implantable rods, pellets, plates or fibers, etc.

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug. Immediate release refers to the release of drug within a short time period following administration, e.g., generally within a few minutes to about 1 to 2 hours.

The phrase “release rate profile” refers to the percentage of active ingredient that is released over fixed units of time, e.g., mcg/hr, mcg/day, mg/hr, mg/day, 10% per day for ten days, and the like. As persons of ordinary skill know, a release rate profile may be but need not be linear. By way of a non-limiting example, the drug depots may be pellets and/or strips that release at least one analgesic agent in a bolus dose and at least one anti-inflammatory agent over a period of time.

The term “biodegradable” includes that all or parts of the drug depots will degrade over time by the action of enzymes, by hydrolytic action and/or by other similar mechanisms in the human body. In various embodiments, “biodegradable” includes that the depots can break down or degrade within the body to non-toxic components after or while a therapeutic agent has been or is being released. By “bioerodible,” it is meant that the depots will erode or degrade over time due, at least in part, to contact with substances found in the surrounding tissue, fluids or by cellular action. By “bioabsorbable,” it is meant that the depots will be broken down and absorbed within the human body, for example, by a cell or tissue. “Biocompatible” means that the depots will not cause substantial tissue irritation or necrosis at the target tissue site.

The depots and/or anchoring members may comprise a non-biodegradable material. Examples of non-biodegradable polymers include, but are not limited to, various cellulose derivatives (carboxymethyl cellulose, cellulose acetate, cellulose acetate propionate, ethyl cellulose, hydroxypropyl methyl cellulose, hydroxyalkyl methyl celluloses, and alkyl celluloses), silicon and silicon-based polymers (such as polydimethylsiloxane), polyethylene-co-(vinyl acetate), poloxamer, polyvinylpyrrolidone, poloxamine, polypropylene, polyamide, polyacetal, polyester, poly ethylene-chlorotrifluoroethylene, polytetrafluoroethylene (PTFE or “Teflon™”), styrene butadiene rubber, polyethylene, polypropylene, polyphenylene oxide-polystyrene, poly-α-chloro-p-xylene, polymethylpentene, polysulfone, non-degradable ethylene-vinyl acetate (e.g., ethylene vinyl acetate disks and poly(ethylene-co-vinyl acetate)), and other related biostable polymers.

Non-resorbable polymers can also include, but are not limited to, delrin, polyurethane, copolymers of silicone and polyurethane, polyolefins (such as polyisobutylene and polyisoprene), acrylamides (such as polyacrylic acid and poly(acrylonitrile-acrylic acid)), neoprene, nitrile, acrylates (such as polyacrylates, poly(2-hydroxy ethyl methacrylate), methyl methacrylate, 2-hydroxyethyl methacrylate, and copolymers of acrylates with N-vinyl pyrrolidone), N-vinyl lactams, polyacrylonitrile, glucomannan gel, vulcanized rubber and combinations thereof. Examples of polyurethanes include thermoplastic polyurethanes, aliphatic polyurethanes, segmented polyurethanes, hydrophilic polyurethanes, polyether-urethane, polycarbonate-urethane and silicone polyether-urethane. Other suitable non-resorbable materials include, but are not limited to, lightly or highly cross-linked biocompatible homopolymers and copolymers of hydrophilic monomers such as 2-hydroxyalkyl acrylates and methacrylates, N-vinyl monomers, and ethylenically unsaturated acids and bases; polycyanoacrylate, polyethylene oxide-polypropylene glycol block copolymers, polygalacturonic acid, polyvinyl pyrrolidone, polyvinyl acetate, polyalkylene glycols, polyethylene oxide, collagen, sulfonated polymers, vinyl ether monomers or polymers, alginate, polyvinyl amines, polyvinyl pyridine, and polyvinyl imidazole. Depending on the amount of crosslinking within the bioresorbable polymers, the degradation time of the polymer can be reduced, thus making the polymer, for the purpose of this invention, appear to be non-resorbable over the time frame of the use of the material for this invention.

The phrase “pain management medication” includes one or more therapeutic agents that are administered to prevent, alleviate or remove pain entirely. These include anti-inflammatory agents, muscle relaxants, analgesics, anesthetics, narcotics and combinations thereof.

In various embodiments, the depots can be designed to cause an initial burst dose of one or more therapeutic agents within the first 24 hours after implantation. “Initial burst” or “burst effect” or “bolus dose” or “pulse dose” refer to the release of therapeutic agent from the depots during the first 24 hours after the depots come in contact with an aqueous fluid (e.g., synovial fluid, cerebral spinal fluid, etc.). The burst effect may be an immediate release. The “burst effect” is believed to be due to the increased release of therapeutic agent from the depots. The initial burst effect or bolus dose may be determined beforehand by formulating the depots by calculating the quotient obtained by dividing (i) the effective amount by weight of therapeutic agent to be released from the depots or regions in a predetermined initial period of time after implantation of the depots, by (ii) the total amount of therapeutic agent that is to be delivered from an implanted composition. It is understood that the initial burst may vary depending on the shape and surface area of the implants.

The burst effect with respect to the regions or depots, in various embodiments, can be designed so that a larger initial dose may be released over a short period of time to achieve the desired effect. For example, if each drug depot is designed to release 15 mg of morphine per 48 hours, then the initial burst dose or bolus dose region will be designed to release a percentage of the dose within the first 24 hours (e.g., 10 mg of morphine or 66% of the 48 hour dose within 24 hours). Thus, the burst effect of each drug depot or region releases more therapeutic agent than the sustained release regions or depots.

Each region or depot that utilizes a burst effect or bolus dose will release more therapeutic agent (e.g., analgesic and/or anti-inflammatory) than a sustained release region or depot. For example, particularly with painful chronic conditions including rheumatoid arthritis, osteoarthritis, a spinal disc herniation (e.g., sciatica), carpal/tarsal tunnel syndrome, lower back pain, lower extremity pain, upper extremity pain, cancer, tissue pain and pain associated with injury or repair of cervical, thoracic, and/or lumbar vertebrae or intervertebral discs, rotator cuff, articular joint, TMJ, tendons, ligaments, muscles, spondilothesis, stenosis, discogenic back pain, and joint pain or the like, the initial burst effect of each drug depot or region of each drug depot will be advantageous as it will provide more immediate pain and/or inflammation relief as a bolus dose of drug will be released at or near the target tissue site and provide the desired reducing, or alleviation of signs or symptoms of pain and/or inflammation. For example, the drug depots collectively or all of the regions of the drug depots collectively may release 51%, 52%, 53%, 54%, 55%, % 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the daily dose within the first one to twelve hours to reduce, prevent or treat pain and/or inflammation. The pain and/or inflammation may also be postoperative pain following surgery.

All or one or more of the drug depots can comprise at least one analgesic agent or its pharmaceutically acceptable salt and/or at least one anti-inflammatory agent or its pharmaceutically acceptable salt may be co-administered with a muscle relaxant. Co-administration may involve administering at the same time in separate drug depots or formulating together the same in each drug depot.

Exemplary muscle relaxants include by way of example and not limitation, alcuronium chloride, atracurium bescylate, baclofen, carbolonium, carisoprodol, chlorphenesin carbamate, chlorzoxazone, cyclobenzaprine, dantrolene, decamethonium bromide, fazadinium, gallamine triethiodide, hexafluorenium, meladrazine, mephensin, metaxalone, methocarbamol, metocurine iodide, pancuronium, pridinol mesylate, styramate, suxamethonium, suxethonium, thiocolchicoside, tizanidine, tolperisone, tubocuarine, vecuronium, or combinations thereof.

All or one or more of the drug depots may also comprise other therapeutic agents or active ingredients in addition to the at least one analgesic agent or its pharmaceutically acceptable salt and at least one anti-inflammatory agent or its pharmaceutically acceptable salt. Suitable additional therapeutic agents include, but are not limited to, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibodies (daclizumab, basilicimab), ABX (anti IL-8 antibodies), recombinant human IL-10, or HuMax IL-15 (anti-IL 15 antibodies).

Other suitable therapeutic agents that may be co-administered with the anti-inflammatory agent and analgesic agent include IL-1 inhibitors, such Kineret® (anakinra) which is a recombinant, non-glycosylated form of the human inerleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors and/or kainate receptors. It is contemplated that where desirable, a pegylated form of the above may be used. Examples of other therapeutic agents include NF kappa B inhibitors such as glucocorticoids and antioxidants such as dithiocarbamate.

Specific examples of additional therapeutic agents suitable for use include, but are not limited to, an anabolic growth factor or anti-catabolic growth factor, an analgesic agent, or an osteoinductive growth factor or a combination thereof.

Suitable anabolic growth or anti-catabolic growth factors include, but are not limited to, a bone morphogenetic protein, a growth differentiation factor, a LIM mineralization protein, CDMP or progenitor cells or a combination thereof.

Suitable analgesic agents include, but are not limited to, acetaminophen, bupivicaine, opioid analgesics such as amitriptyline, carbamazepine, gabapentin, pregabalin, clonidine, opioid analgesics or a combination thereof. Opioid analgesics include, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tramadol or a combination thereof.

For each of the anti-inflammatory agents and analgesic agents, in some embodiments, the release of each compound may be for at least one, at least two, at least three, at least four, at least five, at least six, at least seven, at least eight, at least nine, at least ten, at least eleven, at least twelve, at least thirteen, at least fourteen, or at least fifteen days, or longer.

All or one or more of the drug depots may also be administered with non-active ingredients. These non-active ingredients may have multi-functional purposes including the carrying, stabilizing and controlling the release of the therapeutic agent(s). The sustained release process, for example, may be by a solution-diffusion mechanism or it may be governed by an erosion-sustained process.

In various embodiments, the non-active ingredients will be durable within the tissue site for a period of time equal to (for biodegradable components) or greater than (for non-biodegradable components) the planned period of drug delivery. For example, the depot material may have a melting point or glass transition temperature close to or higher than body temperature, but lower than the decomposition or degradation temperature of the therapeutic agent. However, the pre-determined erosion of the depot material can also be used to provide for slow release of the loaded therapeutic agent(s).

In various embodiments, some or all of the drug depots may not be biodegradable or comprise material that is not biodegradable. Non-biodegradable polymers include, but are not limited to, various cellulose derivatives (carboxymethyl cellulose, cellulose acetate, cellulose acetate propionate, ethyl cellulose, hydroxypropyl methyl cellulose, hydroxyalkyl methyl celluloses, and alkyl celluloses), silicon and silicon-based polymers (such as polydimethylsiloxane), polyethylene-co-(vinyl acetate), poloxamer, polyvinylpyrrolidone, poloxamine, polypropylene, polyamide, polyacetal, polyester, poly ethylene-chlorotrifluoroethylene, polytetrafluoroethylene (PTFE or “Teflon™”), styrene butadiene rubber, polyethylene, polypropylene, polyphenylene oxide-polystyrene, poly-α-chloro-p-xylene, polymethylpentene, polysulfone, non-degradable ethylene-vinyl acetate (e.g., ethylene vinyl acetate disks and poly(ethylene-co-vinyl acetate)), and other related biostable polymers or combinations thereof.

All or one or more of the drug depots may comprise non-resorbable polymers as well. These non-resorbable polymers can include, but are not limited to, delrin, polyurethane, copolymers of silicone and polyurethane, polyolefins (such as polyisobutylene and polyisoprene), acrylamides (such as polyacrylic acid and poly(acrylonitrile-acrylic acid)), neoprene, nitrile, acrylates (such as polyacrylates, poly(2-hydroxy ethyl methacrylate), methyl methacrylate, 2-hydroxyethyl methacrylate, and copolymers of acrylates with N-vinyl pyrrolidone), N-vinyl lactams, polyacrylonitrile, glucomannan gel, vulcanized rubber and combinations thereof. Examples of polyurethanes include thermoplastic polyurethanes, aliphatic polyurethanes, segmented polyurethanes, hydrophilic polyurethanes, polyether-urethane, polycarbonate-urethane and silicone polyether-urethane. Typically, the non-degradable drug depots may need to be removed.

In some instances, it may be desirable to avoid having to remove the drug depots after use. In those instances, all of the depots may comprise a biodegradable material. There are numerous materials available for this purpose and having the characteristic of being able to breakdown or disintegrate over a prolonged period of time when positioned at or near the target tissue. As a function of the chemistry of the biodegradable material, the mechanism of the degradation process can be hydrolytical or enzymatical in nature, or both. In various embodiments, the degradation can occur either at the surface (heterogeneous or surface erosion) or uniformly throughout the drug delivery system depot (homogeneous or bulk erosion).

In various embodiments, the depots may comprise a bioabsorbable, and/or a biodegradable biopolymer that may provide immediate release, or sustained release of the at least one analgesic agent and at least one anti-inflammatory agent. Examples of suitable sustained release biopolymers include but are not limited to poly (alpha-hydroxy acids), poly (lactide-co-glycolide) (PLGA or PLG), polylactide (PLA), polyglycolide (PG), polyethylene glycol (PEG) conjugates of poly (alpha-hydroxy acids), polyorthoesters, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, poly(glycolide-,-caprolactone), -caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof. As persons of ordinary skill are aware, mPEG may be used as a plasticizer for PLGA, but other polymers/excipients may be used to achieve the same effect. mPEG imparts malleability to the resulting formulations.

Where different combinations of polymers are used (bi, tri (e.g., PLGA-PEO-PLGA) or terpolymers), they may be used in different molar ratios, 1:1, 2:1, 3:1, 4:1, 5:1, 6:1, 7:1, 8:1, 9:1, or 10:1. For example, for a 130-day release drug depot, the polymer make up is 50:50 PLGA to 100 PLA. The molecular weight range is 0.45 to 0.8 dI/g.

In various embodiments, the molecular weight of the polymer can be a wide range of values. The average molecular weight of the polymer can be from about 1000 to about 10,000,000; or about 1,000 to about 1,000,000; or about 5,000 to about 500,000; or about 10,000 to about 100,000; or about 20,000 to 50,000.

In some embodiments where the depots comprise at least one biodegradable polymer, the polymer can comprise poly(lactic-co-glycolic acid) (PLA) or poly(orthoester) (POE) or a combination thereof. The poly(lactic-co-glycolic acid) may comprise a mixture of polyglycolide (PGA) and polylactide and in some embodiments, in the mixture, there is more polylactide than polyglycolide. In various other embodiments, there is 100% polylactide and 0% polyglycolide; 95% polylactide and 5% polyglycolide; 90% polylactide and 10% polyglycolide; 85% polylactide and 15% polyglycolide; 80% polylactide and 20% polyglycolide; 75% polylactide and 25% polyglycolide; 70% polylactide and 30% polyglycolide; 65% polylactide and 35% polyglycolide; 60% polylactide and 40% polyglycolide; 55% polylactide and 45% polyglycolide; 50% polylactide and 50% polyglycolide; 45% polylactide and 55% polyglycolide; 40% polylactide and 60% polyglycolide; 35% polylactide and 65% polyglycolide; 30% polylactide and 70% polyglycolide; 25% polylactide and 75% polyglycolide; 20% polylactide and 80% polyglycolide; 15% polylactide and 85% polyglycolide; 10% polylactide and 90% polyglycolide; 5% polylactide and 95% polyglycolide; and 0% polylactide and 100% polyglycolide.

In various embodiments that comprise both polylactide and polyglycolide; there is at least 95% polylactide; at least 90% polylactide; at least 85% polylactide; at least 80% polylactide; at least 75% polylactide; at least 70% polylactide; at least 65% polylactide; at least 60% polylactide; at least 55%; at least 50% polylactide; at least 45% polylactide; at least 40% polylactide; at least 35% polylactide; at least 30% polylactide; at least 25% polylactide; at least 20% polylactide; at least 15% polylactide; at least 10% polylactide; or at least 5% polylactide; and the remainder of the biopolymer being polyglycolide.

In some embodiments, the biodegradable polymer comprises at least 10 wt. %, at least 50 wt. %, at least 60 wt. %, at least 70 wt. %, at least 80 wt. %, at least 85 wt. %, at least 90 wt. %, at least 95 wt. %, or at least 99 wt. % of the formulation. In some embodiments, the at least one biodegradable polymer, the analgesic and the anti-inflammatory are the only components of the depots.

In some embodiments, at least 75% of the particles have a size from about 1 micrometer to about 250 micrometers. In some embodiments, at least 85% of the particles have a size from about 1 micrometer to about 100 micrometers. In some embodiments, at least 95% of the particles have a size from about 1 micrometer to about 30 micrometers. In some embodiments, all of the particles have a size from about 1 micrometer to about 30 micrometers.

In some embodiments, at least 75% of the particles have a size from about 5 micrometer to about 20 micrometers. In some embodiments, at least 85% of the particles have a size from about 5 micrometers to about 20 micrometers. In some embodiments, at least 95% of the particles have a size from about 5 micrometer to about 20 micrometers. In some embodiments, all of the particles have a size from about 5 micrometer to about 20 micrometers.

One or more of the depots may optionally contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfite, sodium bisulfate, sodium thiosulfate, thimerosal, methylparaben, polyvinyl alcohol and phenylethyl alcohol; solubility adjusting agents; stabilizers; and/or cohesion modifiers. Typically, any such inactive materials will be present within the range of 0-75 wt %, and more typically within the range of 0-30 wt %. If the depots are to be placed in the spinal area, in various embodiments, all or one or more of the depots may comprise sterile preservative free material.

The depots can be of different sizes, shapes and configurations, such as for example, strips, rods, sheets, mesh, or the like. There are several factors that can be taken into consideration in determining the size, shape and configuration of the drug depots. For example, both the size and shape may allow for ease in positioning the drug depots at the target tissue site that is selected as the implantation site. In addition, the shape and size of the system should be selected so as to minimize or prevent the drug depots from moving after implantation or injection. In various embodiments, the drug depots can be shaped like pellets, spheres, cylinders such as rods, flat surfaces such as discs, films or sheets, strips, rods, mesh, or the like. For example, in various embodiments, all of the drug depots can be in the shape of a strip. In another embodiment, all of the drug depots can be in the shape of a round film. In still another embodiment, one or more of the depots can be in the shape of a strip and one or more of the depots can be in the shape of a disc. Flexibility may be a consideration so as to facilitate placement of the drug depots. In various embodiments, the drug depots can be different sizes, for example, the drug depots may be a length of from about 2 to 4 cm and width of from about 1-2 cm and thickness of from about 0.25 to 1 mm, or a length of from about 0.5 mm to 5 cm and have a diameter of from about 0.01 to about 2 mm. In various embodiments, the depots can be strips having dimensions of 2.5 cm×1.5 cm×0.5 mm. In various embodiments, the drug depots may have a layer thickness of from about 0.005 to 1.0 mm, such as, for example, from 0.05 to 0.75 mm.

In one exemplary embodiment, a device comprising three square-shaped drug depots is provided. The drug depots all comprise a therapeutically effective amount of a drug capable of releasing the drug over a period of at least one day. The device is packaged such that the drug depots are pre-threaded with a suture with pre-attached needles at its ends. To use the device, a surgeon after accessing the target tissue site following a surgical procedure will thread both of the needles of the suture into the tissue and then tie a knot at both ends of the suture to hold the device in place. The needles can then be removed or the suture can be cut on both ends at a point between each needle and the knot thereby removing the needles. In some embodiments where the device is not pre-threaded with a suture, a surgeon can thread the suture into each of the drug depots through the one or more channels, slits, loops, and/or clips, attach needles to the ends of the suture, then pass the needles and suture through the target tissue site and then anchor the drug depots at or near the target tissue site by tying a knot so as to limit the movement of the drug depot, even in areas where there is excessive blood flow, shifting of tissues during surgical site closure, or great movement of the tissue (e.g., in a joint or muscle area). The needles can then be removed or the suture can be cut on both ends at a point between each needle and the knot thereby removing the needles.

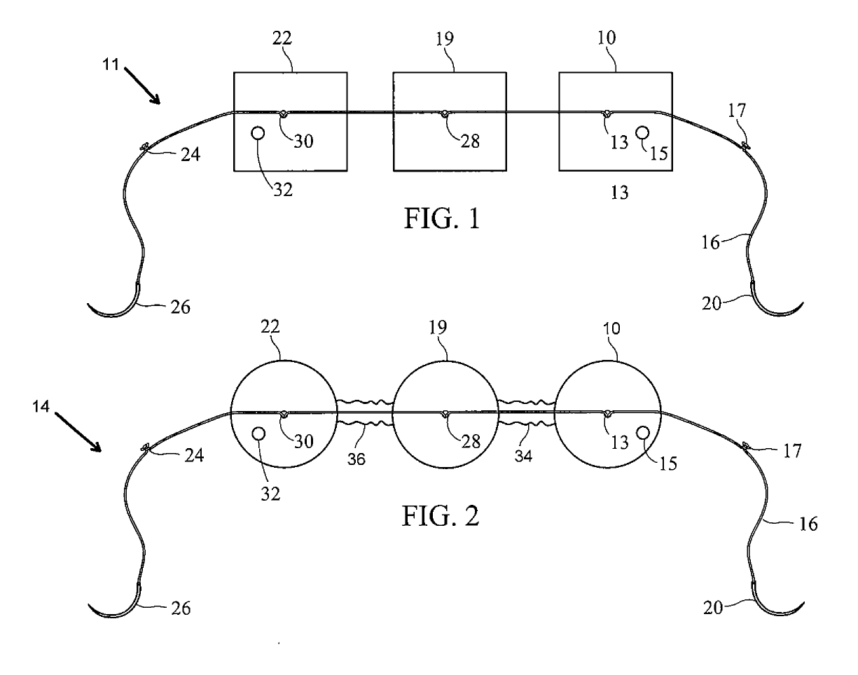

FIG. 1 is a front view of one embodiment of an implantable pre-threaded and pre-knotted device 11. The device 11 has drug depots 10, 19 and 22 connected by a suture 16. The suture 16 comprises a first needle 20 which is pre-attached to the suture 16 at its proximal end and a second needle 26 which is pre-attached to the suture 16 at its distal end. The needles 20 and 26 can pierce tissue at or near a target tissue site. The surface of the target tissue site can be of a sufficient size to receive the suture 16 and needles 20 and 26. Although the drug depots 10, 19 and 22 are shown as square-shaped strips or films, it will be understood by one of ordinary skill in the art that the drug depots 10, 19 and 22 can be of any shape (e.g., round-shaped, triangle-shaped, rectangular-shaped, oval-shaped, etc.) and form (e.g., pellet, rod, sheet, mesh or the like). In some embodiments, the depots 10, 19 and 22 can be spaced 0.5 mm, 1 mm, 5 mm, 10 mm, 20 mm, 50 mm, 100 mm or 1 cm, 5 cm, or 10 cm apart from each other depending on the depth of the target tissue site.

The drug depots 10, 19 and 22 each have a first surface or channel opening 13, 28 and 30, respectively, for receiving the suture 16. It will also be understood by one of ordinary skill in the art that the drug depots 10, 19 and 22 can include one or more ports, grooves, slits, loops, hooks, barbs, posts and/or clips instead of the channel openings 13, 28 and 30 to receive the suture 16. Further, it will be understood by those of ordinary skill in the art that the one or more ports, grooves, slits, loops, hooks, barbs, posts and/or clips can be made of the same or different material than the drug depots 10, 19 and 22. The depots 10 and 22 each also have a second surface or opening 15 and 32, respectively. The second surface 15 of depot 10 receives the first needle 20 and the suture 16 at its proximal end after the surgeon has passed the first needle 20 and suture 16 through the tissue. The second surface 32 of depot 22 receives the second needle 26 and the suture 16 at its distal end after the surgeon has passed the second needle 26 and suture 16 through the tissue.

The suture 16 in FIG. 1 has two regions 17 and 24 that are pre-knotted. The suture regions 17 and 24 are spaced a distance from the depots 10 and 22 to allow the surgeon to place the depots 10, 19 and 22 farther apart if necessary. When the device 11 is placed at the implantation site and the surgeon is ready to lock the suture 16 and device 11 in place, the first needle 20 and the suture 16 at its proximal end are passed through tissue at the implantation site and then the first needle 20 and suture 16 at its proximal end are pulled through the second surface or opening 15 of depot 10 such that the knot 17 is pulled through the tissue and then the second surface or opening 15. The second needle 26 and the suture 16 at its distal end are passed through tissue at the implantation site and then the second needle 26 and suture 16 at its distal end are pulled through the second surface or opening 32 of depot 22 such that the knot 24 is pulled through the tissue and then the second surface or opening 32. After pulling the suture 16 at its proximal and distal ends, all the surgeon needs to do is cut the suture 16 adjacent to the exposed knots 17 and 24 to complete implantation of the device 11 and thereby remove the needles 20 and 26. In various embodiments, this pull and cut system saves the surgeon time consuming-steps of tying knots to anchor the depot. In some embodiments, as the surgeon has no need to tie knots, the drug depot is suitable for laparoscopic, arthroscopic, neuroendoscopic, endoscopic, rectoscopic procedures or the like.

Knots 17 and 24 of the suture 16 have an area larger in circumference and/or diameter than the channels 13, 28 and 30 of the depots and the second surfaces 15 and 30 of depots 10 and 22, respectively. This prevents the device 11 from being able to migrate once it is attached at the implantation site. It will also be understood by those of ordinary skill in the art that the suture 16 and/or knots 17 and 24 can be made of the same or different material as the drug depots 10, 19 and 22.

FIG. 2 is a front view of another embodiment of an implantable pre-threaded and pre-knotted device 14 which is similar to that shown in FIG. 1. The device 14 has drug depots 10, 19 and 22 connected by a suture 16. The suture 16 comprises a first needle 20 which is pre-attached to the suture 16 at its proximal end and a second needle 26 which is pre-attached to the suture 16 at its distal end. The needles 20 and 26 can pierce tissue at or near a target tissue site. The surface of the target tissue site can be of a sufficient size to receive the suture 16 and needles 20 and 26. Although the drug depots 10, 19 and 22 are shown as round or circular-shaped films, it will be understood by one or ordinary skill in the art that the drug depots 10, 19 and 22 can be of any shape (e.g., square-shaped, triangle-shaped, rectangular-shaped, oval-shaped, etc.) and form (e.g., pellet, rod, sheet, mesh or the like). In some embodiments, the depots 10, 19 and 22 can be spaced 0.5 mm, 1 mm, 5 mm, 10 mm, 20 mm, 50 mm, 100 mm or 1 cm, 5 cm, or 10 cm apart from each other depending on the depth of the target tissue site.

The drug depots 10, 19 and 22 each have a first surface or channel opening 13, 28 and 30, respectively, for receiving the suture 16. It will also be understood by one of ordinary skill in the art that the drug depots 10, 19 and 22 can include one or more ports, grooves, slits, loops, hooks, barbs, posts and/or clips instead of the channel openings 13, 28 and 30 to receive the suture 16. Further, it will be understood by those of ordinary skill in the art that the one or more ports, grooves, slits, loops, hooks, barbs, posts and/or clips can be made of the same or different material than the drug depots 10, 19 and 22. The depots 10 and 22 each also have a second surface or opening 15 and 32, respectively. The second surface 15 of depot 10 receives the first needle 20 and the suture 16 at its proximal end after the surgeon has passed the first needle 20 and suture 16 through the tissue. The second surface 32 of depot 22 receives the second needle 26 and the suture 16 at its distal end after the surgeon has passed the second needle 26 and suture 16 through the tissue.

The drug depots 10, 19 and 22 are separated by spacers 34 and 36 to keep the depots 10, 19 and 22 apart or from migrating closer together. It will also be understood by those of ordinary skill in the art that the spacers 34 and 36 can be made of the same or different material as the drug depots 10, 19 and 22 and/or the suture 16. It will also be understood by those of ordinary skill in the art that the shape, form and size of the spacer 34 and 36 can be the same or different than each other and can be of a variety of shapes, forms and sizes dependent upon the particular implantation.

The suture 16 in FIG. 2 has two regions 17 and 24 that are pre-knotted. The suture regions 17 and 24 are spaced a distance from the depots 10 and 22 to allow the surgeon to place the depots 10, 19 and 22 farther apart if necessary. When the device 14 is placed at the implantation site and the surgeon is ready to lock the suture 16 and device 14 in place, the first needle 20 and the suture 16 at its proximal end are passed through tissue at the implantation site and then the first needle 20 and suture 16 at its proximal end are pulled through the second surface or opening 15 of depot 10 such that the knot 17 is pulled through the tissue and then the second surface or opening 15. The second needle 26 and the suture 16 at its distal end are passed through tissue at the implantation site and then the second needle 26 and suture 16 at its distal end are pulled through the second surface or opening 32 of depot 22 such that the knot 24 is pulled through the tissue and then the second surface or opening 32. After pulling the suture 16 at its proximal and distal ends, all the surgeon needs to do is cut the suture 16 adjacent to the exposed knots 17 and 24 to complete implantation of the device 14 and thereby remove the needles 20 and 26.

Knots 17 and 24 of the suture 16 have an area larger in circumference and/or diameter than the channels 13, 28 and 30 of the depots and the second surfaces 15 and 30 of depots 10 and 22, respectively. This prevents the device 14 from being able to migrate once it is attached at the implantation site. It will also be understood by those of ordinary skill in the art that the suture 16 and/or knots 17 and 24 can be made of the same or different material as the drug depots 10, 19 and 22.

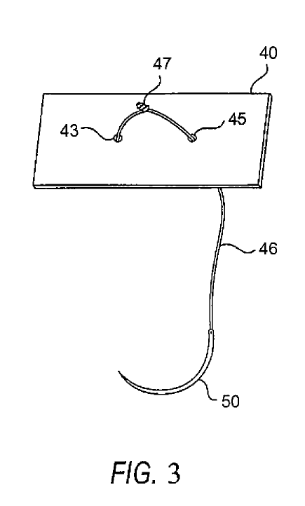

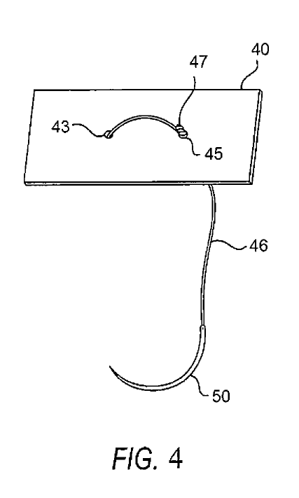

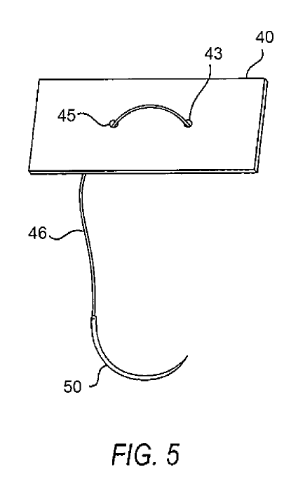

FIGS. 3 and 4 illustrate front views of one embodiment of drug depot 40 of an implantable device as the suture 46 and needle 50 pass through the channel opening 43. FIG. 5 illustrates a back view of this embodiment. The suture 46 has a region 47 (after piercing the body tissue) that the surgeon pulls and forces it through opening 45 and locks the depot 40 in place at or near a target tissue site. This illustration shows the depot 40 pre-threaded and pre-knotted and the suture 46 and needle 50 passed through channel opening 45 and region 47 about to be pulled through (FIGS. 3 and 4). FIG. 5 shows the suture 46 and needle 50 pulled through the channel opening 45 of the drug depot 40. This back part of the depot, in some embodiments, is implanted against the tissue plane.

It will be understood by one of ordinary skill in the art that the depot 40 may have holes, channels, grooves, slits, or the like pre-made in it that is done by the manufacturer. Alternatively, the holes, channels, grooves, slits, or the like can be made by the user using the needle 50.

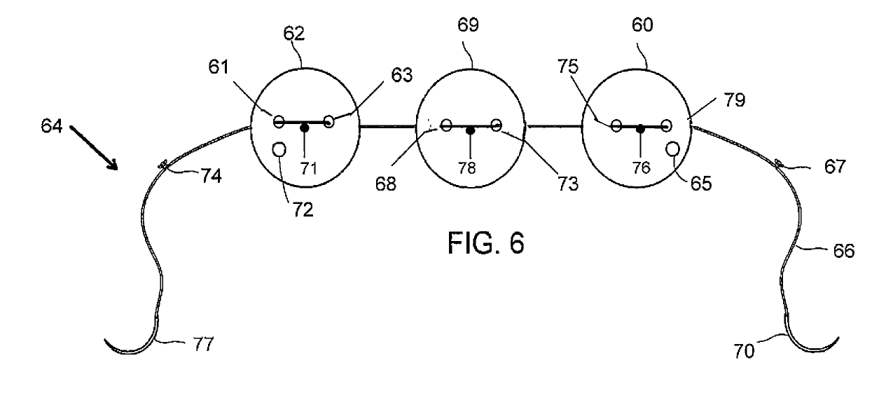

FIG. 6 is a front view of another embodiment of an implantable pre-threaded and pre-knotted device 64. The device 64 has drug depots 60, 69 and 62 connected by a suture 66. The suture 66 comprises a first needle 70 which is pre-attached to the suture 66 at its proximal end and a second needle 77 which is pre-attached to the suture 66 at its distal end. The needles 70 and 77 can pierce tissue at or near a target tissue site. The surface of the target tissue site can be of a sufficient size to receive the suture 66 and needles 70 and 77. Although the drug depots 60, 69 and 62 are shown as round-shaped films, it will be understood by one of ordinary skill in the art that the drug depots 60, 69 and 62 can be of any shape (e.g., square-shaped, triangle-shaped, rectangular-shaped, oval-shaped, etc.) and form (e.g., pellet, rod, sheet, mesh or the like). In some embodiments, the depots 60, 69 and 62 can be spaced 0.5 mm, 1 mm, 5 mm, 10 mm, 20 mm, 50 mm, 100 mm or 1 cm, 5 cm, or 10 cm apart from each other depending on the depth of the target tissue site.

The drug depots 60, 69 and 62 each have a pair of surfaces or channel openings through which the suture 66 is threaded. Suture 66 is threaded through depot 60 through the pair of surfaces or channel openings 79 and 75 such that the suture 66 is behind the depot 60 before it approaches the pair of surfaces or channel openings 79 and 75. The suture 66 has a knot 76 at a point between the pair of surfaces or channel openings 79 and 75. The knot 76 can be made during manufacture of the device 64 and will prevent the depot 60 from migrating much if any from where it is placed.

Depot 69 also has a pair of surfaces or channel openings 73 and 68 similar to depot 60 through which the suture 66 is threaded. The suture 66 has a knot 78 at a point between the pair of surfaces or channel openings 73 and 68. The knot 78 can be made during manufacture of the device 64 and will prevent the depot 69 from migrating much if any from where it is placed.

Depot 62 also has a pair of surfaces or channel openings 63 and 61 similar to depots 60 and 69 through which the suture 66 is threaded. The suture 66 has a knot 71 at a point between the pair of surfaces or channel openings 63 and 61. The knot 71 can be made during manufacture of the device 64 and will prevent the depot 62 from migrating much if any from where it is placed.

It will also be understood by one of ordinary skill in the art that the drug depots 60, 69 and 62 can include a pair of ports, grooves, slits, loops, hooks, barbs, posts and/or clips instead of the pair of surfaces or channel openings 79 and 75, 73 and 68, or 63 and 61 to receive the suture 66. Further, it will be understood by those of ordinary skill in the art that the pair of ports, grooves, slits, loops, hooks, barbs, posts and/or clips can be made of the same or different material than the drug depots 60, 69 and 62. The depots 60 and 62 each also have a third surface or opening 65 and 72, respectively. The third surface 65 of depot 60 receives the first needle 70 and the suture 66 at its proximal end after the surgeon has passed the first needle 70 and suture 66 through the tissue. The third surface 72 of depot 62 receives the second needle 77 and the suture 66 at its distal end after the surgeon has passed the second needle 77 and suture 66 through the tissue.

The suture 66 in FIG. 6 has two regions 67 and 74 that are pre-knotted. The suture regions 67 and 74 are spaced a distance from the depots 60 and 62 to allow the surgeon to place the depots 60, 69 and 62 farther apart if necessary. When the device 64 is placed at the implantation site and the surgeon is ready to lock the suture 66 and device 64 in place, the first needle 70 and the suture 66 at its proximal end are passed through tissue at the implantation site and then the first needle 70 and suture 66 at its proximal end are pulled through the third surface or opening 65 of depot 60 such that the knot 67 is pulled through the tissue and then the third surface or opening 65. The second needle 77 and the suture 66 at its distal end are passed through tissue at the implantation site and then the second needle 77 and suture 66 at its distal end are pulled through the third surface or opening 72 of depot 62 such that the knot 74 is pulled through the tissue and then the third surface or opening 72. After pulling the suture 66 at its proximal and distal ends, all the surgeon needs to do is cut the suture 66 adjacent to the exposed knots 67 and 74 to complete implantation of the device 64 and thereby remove the needles 70 and 77.

Knots 67 and 74 of the suture 66 have an area larger in circumference and/or diameter than the pairs of surfaces or channel openings 79 and 75, 73 and 68, and 63 and 61 and the third surfaces 65 and 72 of depots 60 and 62, respectively. This prevents the device 64 from being able to migrate once it is attached at the implantation site. It will also be understood by those of ordinary skill in the art that the suture 66 and/or knots 67 and 74 can be made of the same or different material as the drug depots 60, 69 and 62.

Knots 76, 78 and 71 have an area larger in circumference and/or diameter than the pairs of surfaces or channel openings 79 and 75, 73 and 68, and 63 and 61 of depots 60, 69 and 62, respectively. The knots 76, 78 and 71 prevent the depots 60, 69 and 62 from being able to migrate much if any once the device 64 is attached at the implantation site. It will also be understood by those of ordinary skill in the art that the knots 76, 78 and 71 can be made of the same or different material as the drug depots 60, 69 and 62.

Radiographic markers can be included on the drug depots to permit the user to track movement and degradation of the depots at the site over time. In this embodiment, the user may accurately position the depots in the site using any of the numerous diagnostic imaging procedures. Such diagnostic imaging procedures include, for example, X-ray imaging, fluoroscopy, or MRI. Examples of such radiographic markers include, but are not limited to, barium, calcium phosphate, and/or metal beads or particles. In various embodiments, the radiographic marker could be a spherical shape, a line(s), or a ring around the depots.

In some embodiments, the drug depots may have an initial burst effect to release the drug shortly after it is implanted. Various factors can be adjusted to achieve the initial burst of therapeutic agent release. First, the initial burst can be controlled by factors related to the properties of the depots, such as the water immiscibility of the solvent, polymer/solvent ratio and the property of the polymer(s). The extent of water immiscibility of the solvent used in the depots affects that rate aqueous body fluid can penetrate the depots to release the therapeutic agent. Generally, higher water solubility leads to a higher initial burst while water immiscibility leads to a lower initial burst or slower release (sustained release) of the therapeutic agent.

Suitable solvents that can be used to control initial burst release or sustained release include, but are not limited to, methyl benzoate, ethyl benzoate, n-propyl benzoate, isopropyl benzoate, butyl benzoate, isobutyl benzoate, sec-butyl benzoate, tert-butyl benzoate, isoamyl benzoate, benzyl benzoate, water, alcohol, low molecular weight PEG (less than 1,000 MW), triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glycerin, ethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, glycofurol, dimethyl sulfoxide, tetrahydrofuran, caprolactam, decylmethylsulfoxide, oleic acid, 1-dodecylazacyclo-heptan-2-one, or mixtures thereof. The solvent can be mixed, in various embodiments, with the therapeutic agent and/or polymers to obtain the desired release profile.

The depots may have pore forming agents, which include biocompatible materials that when contacted with body fluids dissolve, disperse or degrade to create pores or channels in the polymer matrix. Typically, organic and non-organic materials that are water soluble such as sugars (e.g., sucrose, dextrose), water soluble salts (e.g., sodium chloride, sodium phosphate, potassium chloride, and sodium carbonate), water soluble solvents such as N-methyl-2-pyrrolidone and polyethylene glycol and water soluble polymers (e.g., carboxymethylcellulose, hydroxypropyl-cellulose, and the like) can conveniently be used as pore formers. Such materials may be present in amounts varying from about 0.1% to about 100% of the weight of the polymer, but will typically be less than 50% and more typically less than 10-20% of the weight of polymer.

Further, varying the molecular weight of the polymer in the depots, or adjusting the molecular weight distribution of the polymer material in each depot vehicle can affect the initial burst and the release rate of therapeutic agent from the depots. Generally, a higher molecular weight polymer renders a lower initial burst and slower release rate of the therapeutic agent. The polymers may have different end groups such as acid and ester end groups. As persons of ordinary skill in the art are aware, when implantable elastomeric depot compositions having a blend of polymers with different end groups are used, the resulting formulation will have a lower burst index and a regulated duration of delivery. For example, one may use polymers with acid (e.g., carboxylic acid) and ester end groups (e.g., methyl of ethyl ester end groups).

Additionally, by varying the comonomer ratio of the various monomers that form a polymer (e.g., the L/G (lactic acid/glycolic acid) or G/CL (glycolic acid/polycaprolactone) ratio for a given polymer), there will be a resulting depot composition having a regulated burst index and duration of delivery. For example, a depot composition having a polymer with a L/G ratio of 50:50 may have a short duration of delivery ranging from about two days to about one month; a depot composition having a polymer with a L/G ratio of 65:35 may have a duration of delivery of about two months; a depot composition having a polymer with a L/G ratio of 75:25 or L/CL ratio of 75:25 may have a duration of delivery of about three months to about four months; a depot composition having a polymer ratio with a L/G ratio of 85:15 may have a duration of delivery of about five months; a depot composition having a polymer with a L/CL ratio of 25:75 or PLA may have a duration of delivery greater than or equal to six months; a depot composition having a terpolymer of CL/G/L with G greater than 50% and L greater than 10% may have a duration of delivery of about one month and a depot composition having a terpolymer of CL/G/L with G less than 50% and L less than 10% may have a duration months up to six months. In general, increasing the G content relative to the CL content shortens the duration of delivery whereas increasing the CL content relative to the G content lengthens the duration of delivery. Thus, among other things, depot compositions having a blend of polymers having different molecular weights, end groups and comonomer ratios can be used to create depot formulations having a lower burst index and a regulated duration of delivery.

Factors such as the particle size, the disintegration of the particulates, the morphology of the particulates (e.g., whether pores are present in the particulates before implanting or can be formed easily by body fluid attack), coatings, complex formation by the therapeutic agent and the strength of the complex bond can be manipulated to achieve the desired low initial burst and release rate.

### Suture

The suture may be resorbable or permanent in nature depending upon the type of material from which it is made. As used herein, “suture” refers to any flexible structure that can be stretched between two points and includes, without limitation, traditional suture material, single or multiple stranded threads or a mesh structure. A suture may also be a strap-like structure with a number of holes in it, similar to the holes found in a belt. A “suture” may also take the form of an acellular, collagen membrane or other biologic tissue augment, which may provide a scaffold or support matrix for cellular ingrowth to allow soft tissue to reconstruct itself. Sutures for attachment to surgical needles include silk, nylon, linen, cotton, chromic gut, plain gut, cat gut, vicryl, polyglactin, polyester, polypropylene, stainless steel and synthetic polymers having glycolic acid ester linkages subject to hydrolytic degradation to non-toxic tissue compatible absorbable components including polyglycolic acid. The sutures may be monofilamentary or braided, absorbable or non-absorbable. The suture may be of any length provided it is long enough to cover the diameter of multiple drug depots as well as have ends that can reach and pass through the tissue site of placement of the depots. The suture may be of any thickness provided it can be attached to or pass through the drug depots. In some embodiments, the suture may be coated with a drug.

A variety of bioabsorbable polymers can be used to make the suture. Examples of suitable biocompatible, bioabsorbable polymers include aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as collagen, elastin, bioabsorbable starches, etc.) or blends thereof. Polyesters include, but are not limited to, homopolymers and copolymers of lactide (which includes lactic acid, D-,L- and meso lactide), glycolide (including glycolic acid), caprolactone, p-dioxanone (1,4-dioxan-2-one), trimethylene carbonate (1,3-dioxan-2-one), alkyl derivatives of trimethylene carbonate, delta-valerolactone, beta-butyrolactone, gamma-butyrolactone, epsilon-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one 2,5-diketomorpholine, pivalolactone, alpha-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one or polymer blends thereof.

In some embodiments, the suture can comprise shape memory polymers including various polyethers, polyacrylates, polyamides, polysiloxanes, polyurethanes, polyether amides, polyurethane/ureas, polyether esters, or urethane/butadiene copolymers or a combination thereof. In some embodiments, the suture degrades faster than the drug depots. In some embodiments, the drug depots degrade faster than the suture.

Sutures may be of different sizes depending on the procedure being performed and the implant site. Sutures can range in size from #000000 (#6-0 or #6/0), #00 (#2-0 or #2/0), #0, #1, #2, #3, #4, #5, #6, with #000000 being the smallest. In various embodiments, the drug depots will have one or more channels, grooves, slits, loops, hooks, and/or barbs that will be larger than #000000, #00, #0, #1, #2, #3, #4, #5, or #6, range so that the suture can pass through the surface or channel of the drug depot.

### Needles

In some embodiments, a device is provided comprising three or more drug depots connected by a suture, yarn or thread with needles at the ends of the suture, yarn or thread. The drug depots have one or more channels, grooves, slits, loops, hooks, eyelets, barbs, posts and/or clips through which the suture, yarn or thread is received. The suture, yarn or thread is pre-threaded with needles. In some embodiments, the device is not pre-threaded with a suture, yarn or thread and the suture, yarn or thread does not have pre-attached needles. The device includes three or more drug depots having one or more channels, grooves, slits, loops, hooks, eyelets, barbs, posts and/or clips which may be larger than the width and/or thickness of the needles and act as a guide for the surgeon to pass the needles therethrough.

The dimensions of the needles, among other things, will depend on the site for implantation. For example, the width of the muscle planes in different surgical procedures can vary from 1-40 cm. Thus, the needles, in various embodiments, can be designed for these specific areas.

Needles may have different shapes such as for example half curved or ski shaped, ¼ circle, ⅜ circle, ½ circle, ⅝ circle, compound curve or the like.

Suturing needles for applying a device comprising three or more drug depots by hand or via an automated device such as for example in arthroscopic surgeries can be used in the present application. Suturing needles are usually made from a cut blank of material, such as for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. The needles may optionally include one or more tapered regions. In various embodiments, the needles generally include a shaft, a rear end portion with an aperture or channel to secure a suture thread and a needle head at a front end portion for puncturing skin and passing through tissue. The needle head typically incorporates a sharpened needle tip at its distal end and cutting edges. Alternatively, the needle tip may be of a tapered configuration. Straight and curved needles including multiple curved configurations are also known in the art. Suture needles typically incorporate a sharpened needle end. Sharper needles require less force to penetrate tissue and thus cause less tissue trauma. In addition, a sharper needle reduces fatigue on the needle itself, making it less likely to bend or break during suturing. Needle sharpness is typically defined in terms of “penetration force”—the force necessary for a needle to puncture, or penetrate, the tissue. The penetration force is primarily determined by the design and sharpness of the needle point and the cutting edges formed on the needle head. Needle sharpness is also affected by drag force on the needle as it travels through the tissue. The drag force also depends upon the design and sharpness of the needle, and the presence of a lubricating coating. The choice of materials of surgical needle is made to optimize strength, ductility and resistance to bending or breaking of the needle. However, the cross-sectional shape and dimensions of the needle contributes significantly to the physical characteristics of the needle. In various embodiments, the needles include stainless steel such as series “300” stainless steels, which typically have tensile strengths of between 325,000-350,000 lbs/in². When the suture needles are metal such as, for example, stainless steal, the needles can be manufactured through conventional cutting, coining, grinding and/or swaging processes, and may be heat treated to further enhance its strength and resistance to bending. A lubricious coating such as silicon may be applied to a needle body to further enhance penetration and drag characteristics.

### Sterilization

The medical device to administer the drug may be sterilizable. In various embodiments, the drug depots, needles and/or anchoring member (e.g., suture) may be sterilizable by radiation in a terminal sterilization step in the final packaging. Terminal sterilization of a product provides greater assurance of sterility than from processes such as an aseptic process which requires individual product components to be sterilized separately and the final package assembled in a sterile environment.

Typically, in various embodiments, gamma radiation is used in the terminal sterilization step which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In some embodiments, the suture, depots and needles are pre-assembled (including pre-threading and pre-knotted), packaged in a moisture resistant package and then terminally sterilized by gamma irradiation. In use, the surgeon removes the pre-assembled device comprising the drug depots from the sterile package for use.

In various embodiments where the suture, depots and needles are not pre-assembled, electron beam (e-beam) radiation may be used to sterilize one or more components of the device. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity.

Other methods may also be used to sterilize the depots and/or one or more components of the device, including, but not limited to, gas sterilization, such as, for example, with ethylene oxide or steam sterilization.

### Kits

In various embodiments, a kit is provided comprising a device having three or more drug depots with surfaces adapted to receive one or more anchoring members. The kit may include additional parts along with the medical device to be used to implant the medical device. The kit may include the device comprising the drug depots, suture and needles pre-assembled in a first compartment. The second compartment may include gloves, drapes, wound dressings and other procedural supplies for maintaining sterility of the implanting process, as well as an instruction booklet. A third compartment may include additional needles and/or sutures. Each tool may be separately packaged in a plastic pouch that is radiation sterilized. A fourth compartment may include an agent for radiographic imaging. A cover of the kit may include illustrations of the implanting procedure and a clear plastic cover may be placed over the compartments to maintain sterility.

### Administration

In various embodiments, the drug depots may be parenterally administered. The term “parenteral” as used herein refers to modes of administration, which bypass the gastrointestinal tract, and include for example, intramuscular, intraperitoneal, intrasternal, subcutaneous, intra-operatively, intrathecally, intradiskally, peridiskally, epidurally, perispinally, intraarticular or combinations thereof.

In various embodiments, because the analgesic and/or anti-inflammatory agent are locally administered, therapeutically effective doses may be less than doses administered by other routes (oral, topical, etc.). In turn, systemic side effects, such as for example, liver transaminase elevations, hepatitis, liver failure, myopathy, constipation, etc. may be reduced or eliminated.

The drug depots can be delivered to any site beneath the skin, including, but not limited to, at least one muscle, ligament, tendon, cartilage, spinal disc, spinal foraminal space, near the spinal nerve root, or spinal canal.

In various embodiments, a method of treating or preventing postoperative pain or inflammation in a patient in need of such treatment is provided. The method comprises suturing a device at or near the target tissue site wherein the device comprises at least three or more drug depots. Each of the three or more drug depots has a first channel or hole that receives a suture, wherein at least two of the at least three or more drug depots comprise a second channel or hole adapted to receive the suture and a needle after the suture and a needle are passed through the target tissue. Each drug depot is capable of releasing an analgesic and/or anti-inflammatory agent or pharmaceutically acceptable salt thereof over a period of at least 1 day. In some embodiments, the analgesic and/or anti-inflammatory dosage amount and release rate profile are sufficient to reduce inflammation and/or pain for a period of 1-90 days, 1-10 days, 1-3 days, 3-7 days, 3-12 days; 3-14 days, 7-10 days, 7-14 days, 7-21 days, 7-30 days, 7-50 days, 7-90 days, 7-140 days, or 14-140 days.

In some embodiments, the analgesic and/or anti-inflammatory agent or a portion of the analgesic agent and/or anti-inflammatory agent are administered as a bolus dose at the target tissue to provide an immediate release of the analgesic and/or anti-inflammatory agent.

In some embodiments, a device is administered by placement into an open patient cavity during surgery. The drug depots can be placed at positions around the pain generator using a strategy of triangulation which can be effective when administering multiple depot pharmaceutical formulations. It will be understood by one skilled in the art that at least four, at least five, at least six, at least seven, etc., drug depots may be placed around the target tissue site (also known as the pain generator or pain generation site) such that the target tissue site falls within an area whose perimeter is defined by a set of multiple formulations. The active ingredients may then be released from the depots via diffusion in a sustained fashion over a period of time, e.g., 1-3 days, 3-15 days, 5-10 days or 7-10 days post surgery in order to address pain and inflammation.

In some embodiments, the drug depots may release 5%, 10%, 15%, 20%, 25%, 30%, 40%, 50%, 60%, 70%, 80%, 90%, 95%, or 99% of the drug relative to a total amount of the drug loaded in the drug depots over a period of at least three days, at least seven days, at least ten days, at least twenty days, at least thirty days, at least forty days, at least fifty days, at least ninety days, at least one hundred days, at least one-hundred and thirty-five days, at least one-hundred and fifty days, or at least one hundred and eighty days.

### Method of Making

In various embodiments, each drug depot comprising the active ingredients can be made by combining a biocompatible polymer and a therapeutically effective amount of the active ingredients or pharmaceutically acceptable salts thereof and forming the drug depot from the combination.

Various techniques are available for forming at least a portion of each drug depot from the biocompatible polymer(s), therapeutic agent(s), and optional materials, including solution processing techniques and/or thermoplastic processing techniques. Where solution processing techniques are used, a solvent system is typically selected that contains one or more solvent species. The solvent system is generally a good solvent for at least one component of interest, for example, biocompatible polymer and/or therapeutic agent. The particular solvent species that make up the solvent system can also be selected based on other characteristics including drying rate and surface tension.

Solution processing techniques include solvent casting techniques, spin coating techniques, web coating techniques, solvent spraying techniques, dipping techniques, techniques involving coating via mechanical suspension, including air suspension (e.g., fluidized coating), ink jet techniques and electrostatic techniques. Where appropriate, techniques such as those listed above can be repeated or combined to build up the depots to obtain the desired release rate and desired thickness.

In various embodiments, a solution containing solvent and biocompatible polymer are combined and placed in a mold of the desired size and shape. In this way, polymeric regions including barrier layers, lubricious layers and so forth can be formed. If desired, the solution can further comprise one or more of the following: other therapeutic agent(s) and other optional additives such as radiographic agent(s), etc. in dissolved or dispersed form. This results in a polymeric matrix region containing these species after solvent removal. In other embodiments, a solution containing solvent with dissolved or dispersed therapeutic agent is applied to a pre-existing polymeric region, which can be formed using a variety of techniques including solution processing and thermoplastic processing techniques, whereupon the therapeutic agent is imbibed into the polymeric region.

Thermoplastic processing techniques for forming a depot or portions thereof include molding techniques (for example, injection molding, rotational molding, and so forth), extrusion techniques (for example, extrusion, co-extrusion, multi-layer extrusion, and so forth) and casting.

Thermoplastic processing in accordance with various embodiments comprises mixing or compounding, in one or more stages, the biocompatible polymer(s) and one or more of the following: the active ingredients, optional additional therapeutic agent(s), radiographic agent(s), and so forth. The resulting mixture is then shaped into implantable drug depots. The mixing and shaping operations may be performed using any of the conventional devices known in the art for such purposes.

During thermoplastic processing, there exists the potential for the therapeutic agent(s) to degrade, for example, due to elevated temperatures and/or mechanical shear that are associated with such processing. For example, certain therapeutic agents may undergo substantial degradation under ordinary thermoplastic processing conditions. Hence, processing is preferably performed under modified conditions, which prevent the substantial degradation of the therapeutic agent(s). Although it is understood that some degradation may be unavoidable during thermoplastic processing, degradation is generally limited to 10% or less. Among the processing conditions that may be controlled during processing to avoid substantial degradation of the therapeutic agent(s) are temperature, applied shear rate, applied shear stress, residence time of the mixture containing the therapeutic agent, and the technique by which the polymeric material and the therapeutic agent(s) are mixed.

Mixing or compounding a biocompatible polymer with therapeutic agent(s) and any additional additives to form a substantially homogenous mixture thereof may be performed with any device known in the art and conventionally used for mixing polymeric materials with additives.

Where thermoplastic materials are employed, a polymer melt may be formed by heating the biocompatible polymer, which can be mixed with various additives (e.g., therapeutic agent(s), inactive ingredients, etc.) to form a mixture. A common way of doing so is to apply mechanical shear to a mixture of the biocompatible polymer(s) and additive(s). Devices in which the biocompatible polymer(s) and additive(s) may be mixed in this fashion include devices such as single screw extruders, twin screw extruders, banbury mixers, high-speed mixers, ross kettles, and so forth.

Any of the biocompatible polymer(s) and various additives may be premixed prior to a final thermoplastic mixing and shaping process, if desired (e.g., to prevent substantial degradation of the therapeutic agent among other reasons).

For example, in various embodiments, a biocompatible polymer is precompounded with a radiographic agent (e.g., radio-opacifying agent) under conditions of temperature and mechanical shear that would result in substantial degradation of the therapeutic agent, if it were present. This precompounded material is then mixed with therapeutic agent under conditions of lower temperature and mechanical shear, and the resulting mixture is shaped into the active ingredient containing drug depots. Conversely, in another embodiment, the biocompatible polymer can be precompounded with the therapeutic agent under conditions of reduced temperature and mechanical shear. This precompounded material is then mixed with, for example, a radio-opacifying agent also under conditions of reduced temperature and mechanical shear, and the resulting mixture is shaped into the drug depots.

The conditions used to achieve a mixture of the biocompatible polymer and therapeutic agent and other additives will depend on a number of factors including, for example, the specific biocompatible polymer(s) and additive(s) used, as well as the type of mixing device used.

As an example, different biocompatible polymers will typically soften to facilitate mixing at different temperatures. For instance, where depots are formed comprising PLGA or PLA polymer, a radio-opacifying agent (e.g., bismuth subcarbonate), and a therapeutic agent prone to degradation by heat and/or mechanical shear (e.g., clonidine), in various embodiments, the PLGA or PLA can be premixed with the radio-opacifying agent at temperatures of about, for example, 150° C. to 170° C. The therapeutic agent is then combined with the premixed composition and subjected to further thermoplastic processing at conditions of temperature and mechanical shear that are substantially lower than is typical for PLGA or PLA compositions. For example, where extruders are used, barrel temperature, volumetric output are typically controlled to limit the shear and therefore to prevent substantial degradation of the therapeutic agent(s). For instance, the therapeutic agent and premixed composition can be mixed/compounded using a twin screw extruder at substantially lower temperatures (e.g., 100-105° C.), and using substantially reduced volumetric output (e.g., less than 30% of full capacity, which generally corresponds to a volumetric output of less than 200 cc/min). It is noted that this processing temperature is well below the melting points of certain active ingredients, such as an anti-inflammatory and analgesic because processing at or above these temperatures will result in substantial therapeutic agent degradation. It is further noted that in certain embodiments, the processing temperature will be below the melting point of all bioactive compounds within the composition, including the therapeutic agent. After compounding, the resulting depots are shaped into the desired form, also under conditions of reduced temperature and shear.

In other embodiments, biodegradable polymer(s) and one or more therapeutic agents are premixed using non-thermoplastic techniques. For example, the biocompatible polymer can be dissolved in a solvent system containing one or more solvent species. Any desired agents (for example, a radio-opacifying agent, a therapeutic agent, or both radio-opacifying agent and therapeutic agent) can also be dissolved or dispersed in the solvents system. Solvent is then removed from the resulting solution/dispersion, forming a solid material. The resulting solid material can then be granulated for further thermoplastic processing (for example, extrusion) if desired.

As another example, the therapeutic agent can be dissolved or dispersed in a solvent system, which is then applied to pre-existing drug depots (pre-existing drug depots can be formed using a variety of techniques including solution and thermoplastic processing techniques, and it can comprise a variety of additives including a radio-opacifying agent and/or viscosity enhancing agent), whereupon the therapeutic agent is imbibed on or in the drug depots. As above, the resulting solid materials can then be granulated for further processing, if desired.

Typically, an extrusion processes may be used to form the drug depots comprising a biocompatible polymer(s), therapeutic agent(s) and radio-opacifying agent(s). Co-extrusion may also be employed, which is a shaping process that can be used to produce drug depots comprising the same or different layers or regions (for example, a structure comprising one or more polymeric matrix layers or regions that have permeability to fluids to allow immediate and/or sustained drug release). Multi-region depots can also be formed by other processing and shaping techniques such as co-injection or sequential injection molding technology.

In various embodiments, the depots that may emerge from the thermoplastic processing (e.g., pellet, strip, etc.) are cooled. Examples of cooling processes include air cooling and/or immersion in a cooling bath. In some embodiments, a water bath is used to cool the extruded depots. However, where a water-soluble therapeutic agent such as an active ingredient is used, the immersion time should be held to a minimum to avoid unnecessary loss of therapeutic agent into the bath.

In various embodiments, immediate removal of water or moisture by use of ambient or warm air jets after exiting the bath will also prevent re-crystallization of the drug on the depot surfaces, thus controlling or minimizing a high drug dose “initial burst” or “bolus dose” upon implantation or insertion if this is release profile is not desired. Thus, a sustained release region of each drug depot may, in various embodiments, be made by immediately removal of water or moisture.

In various embodiments, the drug depots can be prepared by mixing or spraying the drug with the polymer and then molding the depots to the desired shape. In various embodiments, active ingredients are used and mixed or sprayed with the PLGA or PEG550 polymer, and the resulting depots may be formed by extrusion and dried.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A device for implanting a plurality of drug depots at or near a target tissue site beneath the skin of a patient, the device comprising at least three or more drug depots, each of the at least three or more drug depots having a first surface adapted to receive one or more anchoring members so as to limit movement of the at least three or more drug depots at or near the target tissue site, wherein at least two of the at least three or more drug depots comprise a second surface adapted to receive the anchoring member after the anchoring member contacts the target tissue site, wherein each drug depot is capable of releasing a therapeutically effective amount of the drug over a period of at least one day.

2. A device according to claim 1, wherein the first surface and the second surface comprise one or more channels, holes, ports, grooves, slits, loops, hooks, barbs, posts, tabs and/or clips adapted to receive the one or more anchoring members.

3. A device according to claim 2, wherein each drug depot has a body and the one or more channels, holes, ports, grooves, slits, loops, hooks, barbs, posts and/or clips extends from the body of each drug depot.

4. A device according to claim 1, wherein the one or more anchoring members comprise biodegradable sutures, yarns, threads, lines, wires, staples and/or tacks.

5. A device according to claim 1, wherein each drug depot is biodegradable and the first and second surface comprise one or more channels or holes adapted to receive a biodegradable suture.

6. A device according to claim 5, wherein each drug depot is evenly spaced from each other on the biodegradable suture to provide uniform release of the drug from each drug depot.

7. A device according to claim 5, wherein the biodegradable suture comprises a proximal end and a distal end, the proximal end attached to a first needle for piercing tissue at or near the target tissue site and the distal end attached to a second needle for piercing tissue at or near the target tissue site.

8. A device according to claim 7, wherein the biodegradable suture further comprises a region that surrounds the one or more channels or holes and prevents each drug depot from moving closer or farther from each other.

9. A device according to claim 8, wherein the region of the suture comprises a knot, rim, bead, spacer or clip that prevents each drug depot from moving closer or farther from each other.

10. A device according to claim 5, wherein the device is held in a protective packaging.

11. A device according to claim 1, wherein at least one drug depot has a first surface adapted to receive one or more anchoring members so as to limit movement, but does not have a second surface adapted to receive the anchoring member after the anchoring member contacts the target tissue site.

12. A device according to claim 5, wherein the one or more channels or holes have a portion of the biodegradable suture disposed within the one or more channels or holes so that the biodegradable suture is pre-threaded in the one or more channels or holes.

13. A device according to claim 5, wherein the biodegradable suture comprises a knot, rim, bead or clip disposed near the proximal and the distal end before the first and second needles, the knot, rim, bead or clip configured to pass through and thread the one or more channels of the second surface to hold the at least two drug depots in position at the target tissue site.

14. A device according to claim 1, wherein each drug depot comprises an analgesic and/or anti-inflammatory agent or pharmaceutically acceptable salts thereof and each drug depot is biodegradable and adapted to release the drug over a period of at least 1 day to treat post-operative pain.

15. A device for implanting a plurality of drug depots at or near a target tissue site beneath the skin of a patient, the device comprising at least three or more drug depots, each of the at least three or more drug depots having a first channel or hole adapted to receive one or more sutures so as to limit movement of the at least three or more drug depots at or near the target tissue site, wherein at least two of the at least three or more drug depots comprise a second channel or hole adapted to receive the suture after the suture contacts the target tissue site, wherein each drug depot is capable of releasing a therapeutically effective amount of a drug over a period of at least one day.

16. A device according to claim 15, wherein (i) each drug depot is biodegradable and the first channel or hole receives a biodegradable suture and the second channel or hole receives the same biodegradable suture after the suture contacts the target tissue site so as to anchor the device at or near a target tissue site; (ii) at least one drug depot does not contain a second channel or hole; and (iii) wherein each drug depot is evenly spaced from each other on the biodegradable suture to provide uniform release of the drug from each drug depot.

17. A device according to claim 15, wherein the suture comprises a proximal end and a distal end, the proximal end attached to a first needle for piercing tissue at or near the target tissue site and the distal end attached to a second needle for piercing tissue at or near the target tissue site.

18. A device according to claim 15, wherein the suture further comprises a second region larger than the second channel or hole, the second region of the suture adapted to pass through the second channel or hole when a sufficient pulling force is applied to pull the second region through the second hole or channel to limit movement of the depot at the target tissue site.

19. A method of treating or preventing postoperative pain or inflammation in a patient in need of such treatment, the method comprising suturing a device at or near the target tissue site, the device comprising at least three or more drug depots, each of the three or more drug depots having a first channel or hole that receives a suture, wherein at least two of the at least three or more drug depots comprise a second channel or hole adapted to receive the suture after the suture contacts the target tissue and each drug depot is capable of releasing an analgesic and/or anti-inflammatory agent or pharmaceutically acceptable salt thereof over a period of at least 1 day.

20. A method of treating or preventing postoperative pain or inflammation according to claim 19, wherein the suture further comprises a second region larger than the second channel or hole, the second region of the suture adapted to pass through the second channel or hole when a sufficient pulling force is applied to pull the second region through the second hole or channel to limit movement of the depot at the target tissue site.
